(19) United States
(12) Patent Application Publication
(10) Pub. No.: US 2023/0178221 A1
(43) Pub. Date: Jun. 8, 2023

(54) WHOLE-SLIDE ANNOTATION TRANSFER USING GEOMETRIC FEATURES

(71) Applicant: Ventana Medical Systems, Inc., Tucson, AZ (US)

(72) Inventors: Mohammad Saleh Miri, San Jose, CA (US); Uday Kurkure, Sunnyvale, CA (US)

(73) Assignee: Ventana Medical Systems, Inc., Tucson, AZ (US)

(21) Appl. No.: 18/162,458

(22) Filed: Jan. 31, 2023

Related U.S. Application Data
(63) Continuation of application No. PCT/US21/46827, filed on Aug. 20, 2021.
(60) Provisional application No. 63/069,507, filed on Aug. 24, 2020.

(51) Int. Cl. G16H 30/40; G06T 7/00; G06T 3/40; G06T 7/60; G06V 10/25; G06V 10/44; G06V 10/74

(52) U.S. Cl. CPC G16H 30/40; G06T 7/0012; G06T 3/40; G06T 7/60; G06V 10/25; G06V 10/44; G06V 10/761; G06T 2207/30024

(57) ABSTRACT

A method for transferring digital pathology annotations between images of a tissue sample may include identifying a first set of points for a geometric feature of a first image of a section of a tissue sample; identifying a corresponding second set of points for a corresponding geometric feature of a second image of a same tissue sample, the second image being an image of another section of the tissue sample; determining coordinates of the first set of points and coordinates of the second set of points; determining a transformation between the first set of points and the second set of points; and applying the transformation to a set of digital pathology annotations on the first image to transfer the set of digital pathology annotations within the first image to the second image.

### CROSS-REFERENCE TO RELATED APPLICATIONS

The present application is a continuation of International Application No. PCT/US2021/046827, filed on Aug. 20, 2021, which claims the benefit of and the priority to U.S. Provisional Application No. 63/069,507, filed on Aug. 24, 2020, each of which are hereby incorporated by reference in their entireties for all purposes.

### FIELD

The present disclosure relates to digital pathology, and in particular to techniques for transferring whole slide annotations between images of a tissue sample using geometric features.

### BACKGROUND

Digital pathology involves scanning of the slides (e.g., histopathology or cytopathology glass slides) into digital images. The tissue and/or cells within the digital images may be subsequently examined by digital pathology image analysis and/or interpreted by a pathologist for a variety of reasons including diagnosis of disease, assessment of a response to therapy, and the development of pharmalogical agents to fight disease. In order to examine the tissue and/or cells within the digital images (which are virtually transparent), the pathology slides may be prepared using various stain assays (e.g., immunostains) that bind selectively to tissue and/or cellular components.

One of the most common examples of stain assays is the Hematoxylin-Eosin (H&E) stain assay, which includes two stains that help identify tissue anatomy information. The Hematoxylin mainly stains the cell nuclei with a generally blue color, while the Eosin acts mainly as a cytoplasmic generally pink stain, with other structures taking on different shades, hues, and combinations of these colors. The H&E stain assay may be used to identify target substances in the tissue based on their chemical character, biological character, or pathological character. Another example of example of a stain assay is the Immunohistochemistry (IHC) stain assay, which involves the process of selectively identifying antigens (proteins) in cells of a tissue section by exploiting the principle of antibodies and other compounds (or substances) binding specifically to antigens in biological tissues. In some assays, the target antigen in the specimen to a stain may be referred to as a biomarker. Thereafter, digital pathology image analysis can be performed on digital images of the stained tissue and/or cells to identify and quantify staining for antigens (e.g., biomarkers indicative of tumor cells) in biological tissues.

### SUMMARY

Apparatuses and methods for automatically transferring whole slide annotations between images of a tissue sample using geometric features are provided.

According to various aspects there is provided a method transferring digital pathology annotations between images of a tissue sample. In some aspects, the method may include: identifying a first set of points for a geometric feature of a first image of a section of a tissue sample; identifying a corresponding second set of points for a corresponding geometric feature of a second image of a same tissue sample, the second image being an image of another section of the tissue sample; determining coordinates of the first set of points and coordinates of the second set of points; determining a transformation between the first set of points and the second set of points; and applying the transformation to a set of digital pathology annotations within the first image to transfer the set of digital pathology annotations from the first image to the second image. A minimum number of pairs of points in the first set of points and corresponding points in the second set of points may be three pairs of points. The first set of points and the second set of points may contain the same number of points.

The method may further include converting an area of the section of the tissue sample for the first image and the second image into a grayscale representation to provide a contrast to a background of each image; and identifying the geometric feature based on the contrast between the background of each image and the grayscale representation of the section of the tissue sample.

The method may further include applying a binary mask to an area of the section of the tissue sample for the first image and the second image to provide a contrast to a background of each image; and identifying the geometric feature based on the contrast between the background of each image and the binary mask of the section of the tissue sample.

The method may further include selecting an area containing a portion of the set of digital pathology annotations of the first image having a low magnification; and applying the transformation to the selected area on the first image to transfer the selected area to a corresponding location on the second image.

The method may further include magnifying the first image to a magnification higher than the low magnification to obtain a third image including the selected area; magnifying the second image to a same higher magnification as the third image to obtain a fourth image including the selected area; identifying a third set of points on features within the selected area of the third image; identifying a corresponding fourth set of points on corresponding features within the selected area of the fourth image; determining coordinates of the third set of points on the third image and coordinates of the fourth set of points on the fourth image; determining a transformation between the third set of points and the fourth set of points; and applying the transformation to align a set of digital pathology annotations contained in the selected area of the fourth image to the set of digital pathology annotations contained in the selected area of the third image. A minimum number of pairs of points in the third set of points is three points and corresponding points in the fourth set of points may be three pairs of points. The third set of points and the fourth set of points may contain the same number of points.

The method may further include converting the features within the selected areas of the third image and the fourth image into a grayscale representation to provide a contrast to a background of each image; and identifying specific features based on the contrast between the background of each image and the grayscale representation of the features.

The method may further include applying a binary mask to the features within the selected areas of the third image and the fourth image to provide a contrast to a background of each image; and identifying specific features based on the contrast between the background of each image and the binary mask of the section of the features.

According to various aspects there is provided a non-transitory computer readable medium. In some aspects, the non-transitory computer readable medium may include instructions for causing one or more processors to perform operations for transferring digital pathology annotations between images of a tissue sample, including: identifying a first set of points for a geometric feature of a first image of a section of a tissue sample; identifying a corresponding second set of points for a corresponding geometric feature of a second image of a same tissue sample, the second image being an image of another section of the tissue sample; determining coordinates of the first set of points and coordinates of the second set of points; determining a transformation between the first set of points and the second set of points; and applying the transformation to a set of digital pathology annotations within the first image to transfer the set of digital pathology annotations from the first image to the second image. A minimum number of pairs of points in the first set of points and corresponding points in the second set of points may be three pairs of points. The first set of points and the second set of points may contain the same number of points.

The non-transitory computer readable medium may further include instructions for causing one or more processors to perform operations including converting an area of the section of the tissue sample for the first image and the second image into a grayscale representation to provide a contrast to a background of each image; and identifying the geometric feature based on a contrast between the background of each image and the grayscale representation of the section of the tissue sample.

The non-transitory computer readable medium may further include instructions for causing one or more processors to perform operations including applying a binary mask to an area of the section of the tissue sample for the first image and the second image to provide a contrast to a background of each image; and identifying the geometric feature based on a contrast between the background of each image and the binary mask of the section of the tissue sample.

The non-transitory computer readable medium may further include instructions for causing one or more processors to perform operations including selecting an area containing a portion of the set of digital pathology annotations of the first image having a low magnification; and applying the transformation to the selected area on the first image to transfer the selected area to a corresponding location on the second image.

The non-transitory computer readable medium may further include instructions for causing one or more processors to perform operations including magnifying the first image to a magnification higher than the low magnification to obtain a third image including the selected area; magnifying the second image to a same higher magnification as the third image to obtain a fourth image including the selected area; identifying a third set of points on features within the selected area of the third image; identifying a corresponding fourth set of points on corresponding features within the selected area of the fourth image; determining coordinates of the third set of points on the third image and coordinates of the fourth set of points on the fourth image; determining a transformation between the third set of points and the fourth set of points; and applying the transformation to align a set of digital pathology annotations contained in the selected area of the fourth image to the set of digital pathology annotations contained in the selected area of the third image. A minimum number of pairs of points in the third set of points is three points and corresponding points in the fourth set of points may be three pairs of points. The third set of points and the fourth set of points may contain the same number of points.

The non-transitory computer readable medium may further include instructions for causing one or more processors to perform operations including converting the features within the selected areas of the third image and the fourth image into a grayscale representation to provide a contrast to a background of each image; and identifying specific features based on the contrast between the background of each image and the grayscale representation of the features.

The non-transitory computer readable medium may further include instructions for causing one or more processors to perform operations including applying a binary mask to the features within the selected areas of the third image and the fourth image to provide a contrast to a background of each image; and identifying specific features based on the contrast between the background of each image and the binary mask of the section of the features.

Numerous benefits are achieved by way of the various embodiments over conventional techniques. For example, the various embodiments provide methods and systems that can be used to automatically transfer pathologist digital pathology annotations between image of sequential sections of a tissue sample. In some embodiments, points on the boundary of a tissue sample are identified and used to align the sequential images. A transformation matrix between the identified points can be generated and applied to the annotations. These and other embodiments along with many of its advantages and features are described in more detail in conjunction with the text below and attached figures.

### BRIEF DESCRIPTION OF THE DRAWINGS

Aspects and features of the various embodiments will be more apparent by describing examples with reference to the accompanying drawings, in which:

FIG. 1 illustrates images of serial sections of a tissue sample with digital pathology annotations on the first image;

FIG. 2 illustrates images of serial sections of a tissue sample with digital pathology annotations according to some aspects of the present disclosure;

FIG. 3 illustrates areas of digital pathology annotations on an image of a tissue sample at low magnification according to various aspects of the present disclosure;

FIG. 4 illustrates a illustrates a misalignment of a transferred area of digital pathology annotations under high magnification according to various aspects of the present disclosure;

FIG. 5 illustrates the aligned transferred area of digital pathology annotations of FIG. 4 under high magnification according to various aspects of the present disclosure;

FIG. 6 is a flowchart illustrating an example of a method 600 for transferring digital pathology annotations between images according to some aspects of the present disclosure;

FIG. 7 is a flowchart illustrating an example of a method 700 for transferring digital pathology annotations of selected areas between images according to some aspects of the present disclosure; and

FIG. 8 is a block diagram of an example computing environment with an example computing device suitable for use in some example implementations.

### DETAILED DESCRIPTION

While certain embodiments are described, these embodiments are presented by way of example only, and are not intended to limit the scope of protection. The apparatuses, methods, and systems described herein may be embodied in a variety of other forms. Furthermore, various omissions, substitutions, and changes in the form of the example methods and systems described herein may be made without departing from the scope of protection.

### I. Overview

Evaluation of tissue changes caused, for example, by disease, may be performed by examining thin tissue sections. Tissue samples may be sliced to obtain a series of sections (e.g., 4-5 μm sections), and each tissue section may be stained with different stains or markers to express different characteristics of the tissue. Each section may be mounted on a slide and scanned to create a digital image for examination by a pathologist. The pathologist may review and manually annotate the digital image of the slides (e.g., tumor area, necrosis, etc.) to enable extracting meaningful quantitative measures using image analysis algorithms. Conventionally, the pathologist would manually annotate each successive image of tissue sections from a tissue sample to identify the same aspects on each successive tissue section.

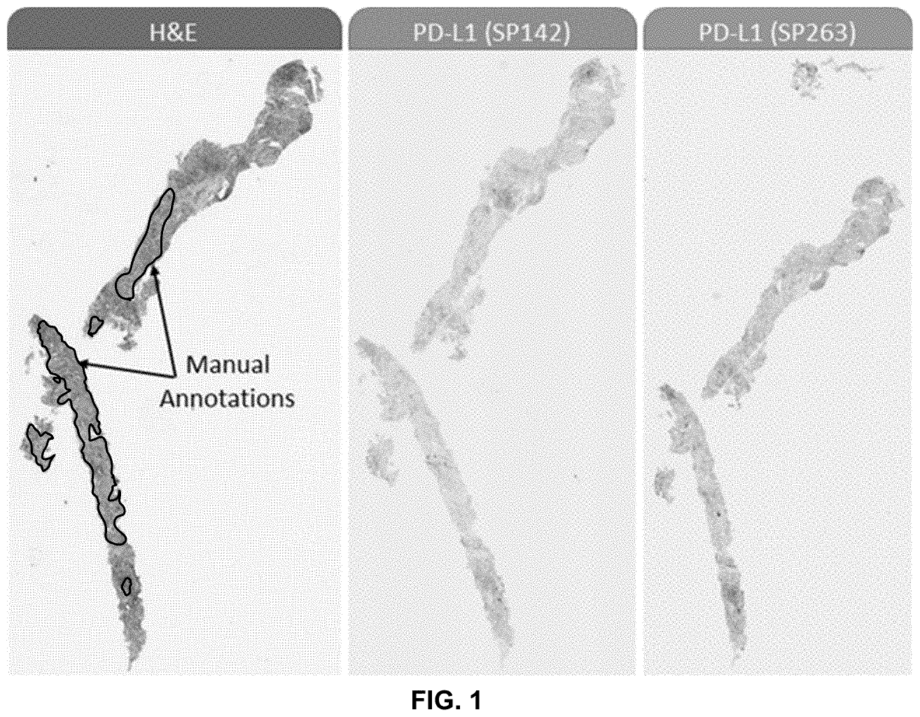

FIG. 1 illustrates images of serial sections of a tissue sample with digital pathology annotations on the first image. As shown in FIG. 1, the serial sections of tissue were stained using H&E, PD-L1 SP142 and PD-L1 SP263 biomarkers and scanned with different slide scanners. Conventionally, a pathologist would manually annotate the first (H&E) image identifying which part of the tissue (e.g., tumor regions, necrotic regions, etc.) to be analyzed using image analysis, as well as regions to be excluded from the image analysis. The pathologist would then manually reproduce the digital pathology annotations on each preceding or subsequent image (PD-L1 SP142 and PD-L1 SP263) individually in order to enable automated image analysis. The repeated annotating of the images of serial sections of tissue consumes a large amount of the pathologist's time.

In order to overcome these limitations as well as others, embodiments of the present disclosure provide for the automated transfer of whole slide annotations between images of a tissue sample using geometric features. The annotation transfer process includes obtaining images of a tissue sample (e.g., tissue and/or cell slide images) and digital pathology annotations for at least one image (e.g., a source image) of the images, aligning pairs of images (e.g., the source image and one or more successive target images) using a feature-based registration technique, and transferring the digital pathology annotations from the source image to the successive target images based on the alignment of the images. The annotation transfer process may be stain and scanner agnostic. Registration of images of slides for different stain assays (e.g., H&E or IHC) that are acquired by different types of scanners (e.g., different scanners from different equipment vendors or different versions of a same scanner from a same equipment vendor) may enable annotation transfer between the slide images. The feature-based registration technique relies on finding corresponding points between source and target images; however, stain assay features of images (e.g., a group of pixels with a similar pixel intensity representative of a fluorescing antigen) cannot be used for finding the corresponding points when source and target images are from two different types of stain assays (e.g., IHC and HE) or both stain assays contain stains targeting different morphological structures (e.g., different IHC antigens). In such instances, embodiments of the present disclosure align the pairs of sample images based on the geometric features (e.g., corners, curvatures, etc.) of the contour of tissue rather than its content, e.g., the stained cells with IHC assays. Geometric features are features of objects constructed by a set of geometric elements such as points, lines, or curves that can be detected by feature detection methods. A transformation is computed from corresponding points (which are geometric entities as opposed to color/intensity) in the two images. These points are computed from a feature image which can be appearance based (e.g., gray-scale intensity image) or geometric features based (e.g. boundary contour, line segments etc.). For example, the contour of a depiction of the tissue and/or cell in the sample images, rather than the stain assay features of images, may provide geometric features (e.g., corners, curvatures, etc.) or appearance based features (e.g., grayscale intensity images) on which alignment may be based. This alignment means that the same tissue and/or cell structures on two matching images correspond with each other spatially.

### II. Definitions

As used herein, when an action is “based on” something, this means the action is based at least in part on at least a part of the something.

As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent.

As used herein, the term “sample” “biological sample” or “tissue sample” refers to any sample including a biomolecule (such as a protein, a peptide, a nucleic acid, a lipid, a carbohydrate, or a combination thereof) that is obtained from any organism including viruses. Other examples of organisms include mammals (such as humans; veterinary animals like cats, dogs, horses, cattle, and swine; and laboratory animals like mice, rats and primates), insects, annelids, arachnids, marsupials, reptiles, amphibians, bacteria, and fungi. Biological samples include tissue samples (such as tissue sections and needle biopsies of tissue), cell samples (such as cytological smears such as Pap smears or blood smears or samples of cells obtained by microdissection), or cell fractions, fragments or organelles (such as obtained by lysing cells and separating their components by centrifugation or otherwise). Other examples of biological samples include blood, serum, urine, semen, fecal matter, cerebrospinal fluid, interstitial fluid, mucous, tears, sweat, pus, biopsied tissue (for example, obtained by a surgical biopsy or a needle biopsy), nipple aspirates, cerumen, milk, vaginal fluid, saliva, swabs (such as buccal swabs), or any material containing biomolecules that is derived from a first biological sample. In certain embodiments, the term “biological sample” as used herein refers to a sample (such as a homogenized or liquefied sample) prepared from a tumor or a portion thereof obtained from a subject.

As used herein, the term “biological material or structure” refers to natural materials or structures that comprise a whole or a part of a living structure (e.g., a cell nucleus, a cell membrane, cytoplasm, a chromosome, DNA, a cell, a cluster of cells, or the like).

As used herein, the term “non-target region” refers to a region of an image having image data that is not intended to be assessed in an image analysis process. Non-target regions may include non-tissue regions of an image corresponding to a substrate such as glass with no sample, for example where there exists only white light from the imaging source. Non-target regions may additionally or alternatively include tissue regions of an image corresponding to biological material or structures that are not intended to be analyzed in the image analysis process or difficult to differentiate from biological material or structures within target regions (e.g., necrosis, stromal cells, normal cells, scanning artifacts).

As used herein, the term “target region” refers to a region of an image including image data that is intended be assessed in an image analysis process. Target regions include any region such as tissue regions of an image that is intended to be analyzed in the image analysis process (e.g., tumor cells or staining expressions).

As used herein, the term “tile” or “tile image” refers to a single image corresponding to a portion of a whole image, or a whole slide. In some embodiments, “tile” or “tile image” refers to a region of a whole slide scan or an area of interest having (x,y) pixel dimensions (e.g., 1000 pixels by 1000 pixels). For example, consider a whole image split into M columns of tiles and N rows of tiles, where each tile within the M×N mosaic comprises a portion of the whole image, i.e. a tile at location MI,NI comprises a first portion of an image, while a tile at location M3,N4 comprises a second portion of the image, the first and second portions being different. In some embodiments, the tiles may each have the same dimensions (pixel size by pixel size).

### III. Techniques for Automated Image Registration

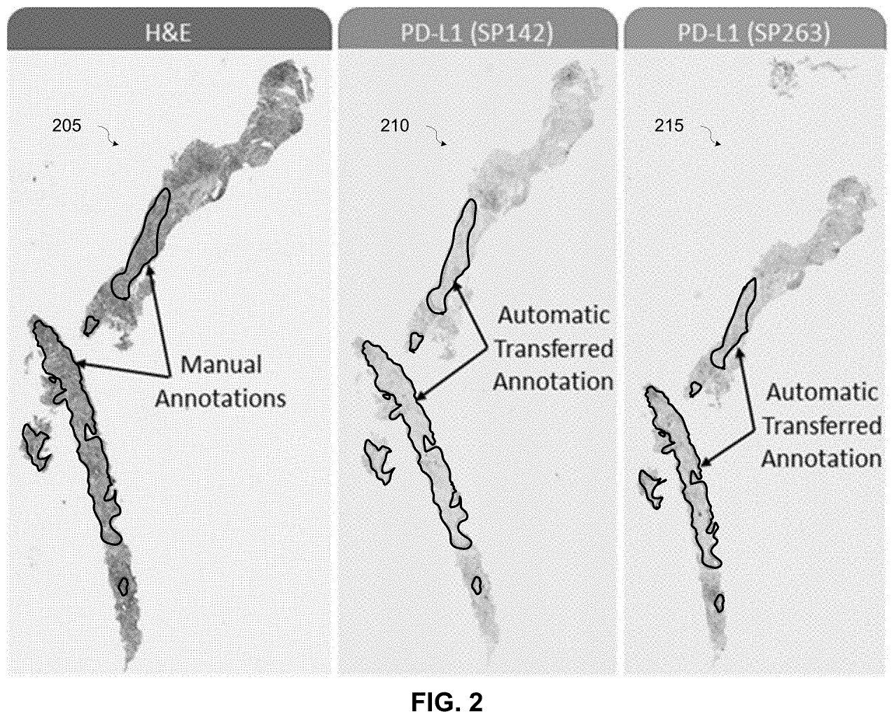

FIG. 2 illustrates images of serial sections of a tissue sample with digital pathology annotations according to some aspects of the present disclosure. As shown in FIG. 2, serial sections of a tissue sample are stained using multiple stain assays for different structures and biomarkers. For example, a first section 205 of the tissue sample may be stained with H&E stain and successive sections 210; 215 of the tissue sample may be stained with one or more IHC stains (e.g., PD-L1 SP142 and PD-L1 SP263). The first section 205 and successive sections 210; 215 of the tissue sample may be scanned using one or more scanners to obtain images of tissue and/or cells within the tissue samples. The one or more scanners may be the same scanner, different versions of the same scanner, or different types of scanners (e.g., a Aperio AT2 brightfield scanner and a Ventana® DP 200 brightfield scanner).

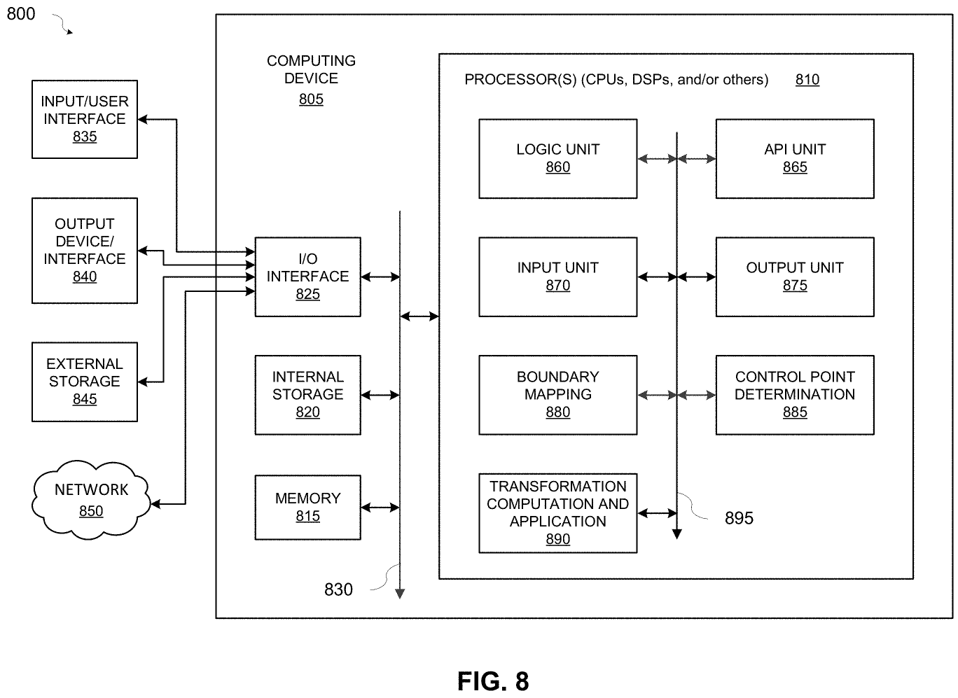

A source image of the first section 205 may be selected as representative of the tissue sample and is annotated manually by the pathologist. As described in detail herein, embodiments of the present disclosure automatically transfer the manual annotations from the source image to preceding or subsequent target images of successive sections 210; 215 of the tissue sample based on the alignment of the source and target images. Aspects of the present disclosure can align the source and target images of tissue sections via an image registration process that includes: 1) finding a corresponding magnification level between the image pyramids associated with each of the source and target images; 2) computing a feature image; 3) localizing control points for an image; 4) finding matching control points between images; and 5) computing a transformation between the images using the inlier control points. An image registration algorithm executing on a computer system (e.g., the computer system of FIG. 8) may execute the above operations.

In order to align source and target tissue section images, a corresponding magnification or resolution level between the image pyramids associated with each of the source and target images may be determined. Whole slide scanners capture images of tissue sections tile by tile or in a line-scanning fashion. The multiple images (tiles or lines, respectively) are captured and digitally assembled (“stitched”) to generate a digital image of the entire slide. An image pyramid is a multi-resolution representation of the digital image of the entire slide. Whole slide images are stored at multiple resolutions to accommodate loading and rendering of the images. For example, a whole slide image acquired at 40× magnification by a slide scanner may be accompanied by the same image downsampled at 10×, 2.5×, and 1.25× magnifications. The low magnification images may be advantageously used for analysis such as image registration because these images require less memory for processing as compared to the high magnification images, and once source and target tissue section images are aligned, the digital pathology annotations may be transferred for target tissue section images using the high magnification images.

However, since images may be acquired using different scanners, the image pyramids associated with each of the source and target may come in different formats. Some image formats (e.g., .SVS) do not follow a consistent approach to building the image pyramid, whereas other formats (e.g., binary information file (.BIF)) follow a consistent approach for building the image pyramid. For example, the second level of the image pyramid in the .BIF format stores an image at 10× magnification. On the other hand, the number of pyramid levels and the magnification at each level are not consistent for .SVS format images. The second level of the image pyramid in the .SVS format could store an image at any magnification or resolution. This inconsistency in building the image pyramids makes it difficult to identify images of the same magnification or resolution for image registration (e.g., level 2 of each pyramid is not necessarily always 10× magnification). Consequently, a corresponding magnification or resolution level between the image pyramids associated with each of the source and target images may be determined.

In order to determine the corresponding magnification or resolution level between the image pyramids associated with each of the source and target images, the image pyramid associated with the source may be analyzed to determine magnification or resolution at each level of the image pyramid, and the image pyramid associated with the target may be analyzed to determine magnification or resolution at each level of the image pyramid. The determined magnification or resolution at each level of the image pyramid for the source image may then be compared to the determined magnification or resolution at each level of the image pyramid for the target image. A corresponding magnification or resolution level between the image pyramids associated with each of the source and target images is identified based on the comparison. For example, if the determined magnification or resolution at a third level of the image pyramid for the source image is 15× and the determined magnification or resolution at a second level of the image pyramid for the target image is 15×, then the corresponding magnification level to be used may be identified as 15× based on the comparison and match between magnification levels. In other instances, the corresponding magnification or resolution level between the image pyramids associated with each of the source and target images is identified based on the comparison and a threshold magnification or resolution level. For example, low magnification or resolution images may be used for the image registration, and thus pairs of images to be used in the alignment process may be thresholded at a maximum magnification or resolution level, e.g., 10× magnification. Consequently, if the determined magnification or resolution at a second level of the image pyramid for the source image is 10× and the determined magnification or resolution at a third level of the image pyramid for the target image is 10×, then the corresponding magnification or resolution level to be used may be identified as 10× based on the comparison and match between magnification or resolution levels and the 10× magnification or resolution threshold.

Once the corresponding magnification or resolution level for the source and target images has been determined, feature image may be determined for the source and target images at the corresponding magnification or resolution level. Point features (features extracted around control or interest points), as the basis of lines, surfaces, and bodies, may be used in the image registration. To obtain a spatial transformation of point features, many point set matching algorithms (PMs) have been developed to match two point sets by optimizing various distance functions. However, when source and target images are from two different types of scanning assays (e.g., IHC and H&E), or both images are IHC staining assays but contain different stains (e.g., PD-L1 SP142, PD-L1 SP263), matching points between the images may not be obtained from the features specific to the staining of the tissue and/or cells such as points, edges or objects with related or contrasting pixel intensities. The different stain assays (e.g., HE and IHC) produce different colors that can cause images to have different features specific to the staining of the tissue and/or cells (e.g., in one staining assay the nucleus of cell may be blue, whereas in another staining assay the same nucleus may be almost transparent). Therefore, pixel values of the features specific to the staining of the tissue and/or cells may not be usable to extract control points for aligning point features and images.

Conversely, the overall shape of portions of the tissue and/or cells at a corresponding magnification or resolution level may be substantially constant between images obtained from different staining assays, different stains, or different scanning equipment. Therefore, geometric features such as the overall shape of portions of the tissue and/or cells may be usable to identify control points for extracting features and aligning images. Aspects of the present disclosure utilize feature images to obtain matching points between images. The feature images are fundamentally the source and target images at the corresponding magnification or resolution level modified to highlight or emphasize the geometric features such as the overall shape of portions of the tissue and/or cells. In some instances, feature images generated for source and target images of the same type of stain assay and stain may be grayscale versions of the source and target images emphasizing the geometric features such as contour, or boundary, of portions of the tissue and/or cells within the images. Grayscale is a range of monochromatic shades from black to white. Therefore, a grayscale image contains only shades of gray and no color, which in some instances filters out noise from color channels that could make it difficult to discern geometric feature within an image. In other instances, feature images generated for source and target images of different types of stain assay or stains or from different image scanners may be a binary mask emphasizing the geometric features such as contour, or boundary, of the tissue and/or cells within the image. A binary mask is a binary raster that contains pixel values of 0 and 1, for example, 0s assigned to pixels identified as background and is assigned to pixels identified as tissue and/or cells. The binary mask may provide contrast between the image background and the boundary of the tissue section, which in some instances filters out noise that could make it difficult to discern geometric feature within an image. In some implementations, a user may choose the type of feature image (e.g., greyscale or binary mask) to be used for the image registration. In other implementations, the type of feature image may be determined automatically, for example, by a computer executing the image registration algorithm.

As discussed herein, the idea behind using the gray scale image or binary mask for finding matching points between source and target images is that when the features specific to the staining of the tissue and/or cells are not suitable for finding the matching points, the geometric features of the tissue and/or cells can be leveraged for alignment. Since the source and target images are sequential thin sections of the same tissue sample, the geometric features of the tissue and/or cells may remain substantially constant between the images; therefore, the gray scale image or binary mask of the tissue can carry the geometric feature (e.g., boundary) information of the tissue and/or cells for the successive images. Thus, the feature image may utilize either the entire tissue section (e.g., feature image=grayscale image) or only the geometric feature (e.g., feature image=binary mask) of the tissue section for aligning the source and target images. Other types of feature images, for example, but not limited to, edge feature images, entropy feature images, etc., may be used without departing from the scope of the present disclosure.

In some instances, the feature maps for each image of the pair of images (i.e., source and target images) having the corresponding magnification or resolution level are generated by converting the color image to black and white, or grayscale. This process removes all color information, leaving only the luminance of each pixel. Since digital color images are displayed using a combination of red, green, and blue (RGB) colors, each pixel has three separate luminance values. Therefore, these three values will be combined into a single value when removing color from an image. There are several ways to do this. In some instances, all luminance values for each pixel are averaged. In other instances, only the luminance values from the red, green, or blue channel are kept. In yet other instances, a grayscale conversion algorithm may be used that allows for conversion of the luminance values from the color channels to generate a black and white image.

In some instances, the feature maps for each image of the pair of images (i.e., source and target images) having the corresponding magnification or resolution level are generated by image segmentation and mask generation. Image segmentation identifies non-target regions and target regions of an image, e.g., distinguishes between background and tissue. One technique for image segmentation may be image thresholding, which generates a binary image from a single band or multi-band image. The image thresholding includes selecting one or more threshold levels that distinguish between pixels in the background and pixels in the tissue and assigning all pixel values above/below a given threshold map to zero (e.g., black) and all pixel values above/below a given threshold map to one (e.g., white). The one or more thresholds may be selected using several methods including the maximum entropy method, balanced histogram thresholding, Otsu's method (maximum variance), k-means clustering, or combinations thereof. Other techniques that may be used for image segmentation include clustering techniques (e.g., K-means algorithm is an iterative technique that is used to partition an image into K clusters), histogram-based techniques (e.g., a histogram is computed from all of the pixels in the image, and the peaks and valleys in the histogram are used to locate the clusters in the image), edge detection techniques, regional growing techniques, partial differential equation (PDE)-based techniques, and the like. The image segmentation creates a pixel-wise mask for each object (e.g., tissue and/or cells in the image providing a more granular understanding of the geometric feature (e.g., boundary) information of the object(s) in the image.

Once the feature images are generated, features are detected within the feature images using a feature detector and describer. Some of the lowest-level features to be detected in an image are the specific positions of some distinguishable points such as corners, edge points, or straight line points. These distinguishable points are known as control points or interest points. As used herein, a “control point” or “interest point” is a member of a set of points which are characterized by a mathematically well-founded definition that can be used to determine the geometric features such as shape or contour of an object within an image. The control points (e.g., the corners which appear at the intersection of two or more image edges) have specific characteristics including: a clearly defined position in the image space, they are rich in terms of information content (e.g., local image structure around the control point is rich in terms of local information contents such as significant 2D texture, and they are also stable on local and global changes in the image domain (e.g., stable under local and global perturbations in the image domain as illumination/brightness variations, such that the interest points can be reliably computed with high degree of repeatability). The control points can be used as good indicators of the geometric features (e.g., boundaries) of the image sequences and can be matched between successive images such as a source and target image. A large number of detected control points in the images increases the possibility of matching points between successive images and the likelihood of successful registration (alignment) of the images.

The control points are generally detected in the form of corners, blob points, edge points, junctions, line points, curve points, etc. The detected control points are subsequently described in logically different ways on the basis of unique patterns possessed by their neighboring pixels. This process is called feature description as it describes each control point assigning it a distinctive identity which enables their effective recognition for matching. Some feature-detectors are available with a designated feature description algorithm while others exist individually. However the individual feature-detectors can be paired with several types of pertinent feature-descriptors. Scale-invariant feature transform (SIFT), Speed Up Robust Features (SURF), Features from Accelerated Segment Test (FAST), KAZE, Accelerated-KAZE (AKAZE), Oriented FAST and Rotated BRIEF (ORB), and Block Regional Interpolation Scheme for K-Space (BRISK) are among the fundamental scale, rotation and affine invariant feature-detectors, each having a designated feature-descriptor and possessing its own advantages and limitations.

After feature detection and description, feature matching is performed between the source and target images based on the detected and described control points. The feature matching establishes a one-to-one correspondence (i.e., matching) between control points on the source image and the control points on the target image. In order to find the one-to-one correspondence between control points, features from the neighborhood of each control point are extracted to characterize the local appearance of each the neighborhood. The basic idea behind the feature extraction is characterizing the local appearance of each control point's neighborhood. The features extracted from the neighborhood of each control point may then be compared to one another to identify the closest matching (e.g., inlier) control points between the source and target images. A number of standard feature computation methods, for example, Histogram of Oriented Gradients (HOG), SURF, SIFT, etc., may be used for feature extraction and different matching strategies can be adopted for matching features such as threshold based matching, nearest neighbor, nearest neighbor distance ratio, and the like. For example, in the instance of generating a binary mask of tissue and/or cells as the feature image, since the available information is limited to the geometric feature such as the shape of the tissue, a HOG technique may be used as the feature extractor to capture the distribution of local gradients or edge directions around each control point. The distribution of local gradients or edge directions for each control point may then be compared to one another via threshold based matching, nearest neighbor, nearest neighbor distance ratio, and the like to identify matching control points. Further, since features extracted by different computation methods or techniques (e.g., HOG versus SURF) provide different localized information for each control point, the feature matching may be implemented as an combinational approach to identify matching control points between source and target images. For example, the HOG computation method may be used to extract the distribution of local gradients or edge directions around each control point, which can then be used to identify matching control points, and one or more additional methods, for example, SURF, SIFT, etc., may be used to extract other features (e.g., string based descriptors or hamming distance) around each control point, which can be used to confirm matching control points and/or identify additional matching control points.

After the matching control points (i.e., inlier control points on the target and source) are identified, coordinates of the matching control points may be determined for the source image and the target image. A transformation matrix between the matching control points on the source image and the control points on the target image may be computed using the coordinates of the control points. However, inaccuracies in matching control points (or outliers) cannot be completely avoided in the feature matching, and can result in generation of an incorrect transformation matrix. The Random Sample Consensus (RANSAC), M-estimator Sample Consensus (MSAC), and Progressive Sample Consensus (PROSAC) are some probabilistic methods or techniques that can be utilized for removing the outliers from matched features and fitting the transformation function (in terms of the transformation matrix). For example, the RANSAC method can be used to filter out the outlier matches and use the inlier matches to fit the transformation function and compute the transformation matrix.

The transformation matrix may be a similarity transform matrix. A similarity transform matrix may provide registration (alignment) between source and target images using translation, rotation, and scaling. A minimum of three pairs of control points between the source and target may be used to compute the transform matrix. More than three pairs of control points may be used when additional pairs of control points are available. A larger number of pairs of control points may increase the accuracy of the alignment. In addition, various metrics may be evaluated to determine the quality of the control points, and only the control points exceeding a specified quality threshold may be used for computing the transformation matrix.

The threshold may be set empirically, and may contribute to the tolerance for the registration error (e.g., the amount of acceptable misalignment). The RANSAC method may be used to remove low-quality matches while computing the transformation matrix. Thus, the transformation that provides the most accurate alignment between two images may be kept while low-quality matches may appear as outliers for the final transformation.

The transformation matrix may be applied to the annotations on the source image to transfer the annotations to the target image. FIG. 2 illustrates an example of the digital pathology annotations automatically transferred from an H&E source image to a PD-L1 (SP142) target image. The process may be repeated with PD-L1 (SP142) image as the source image to transfer the annotations to the PD-L1 (SP263) image as the target image.

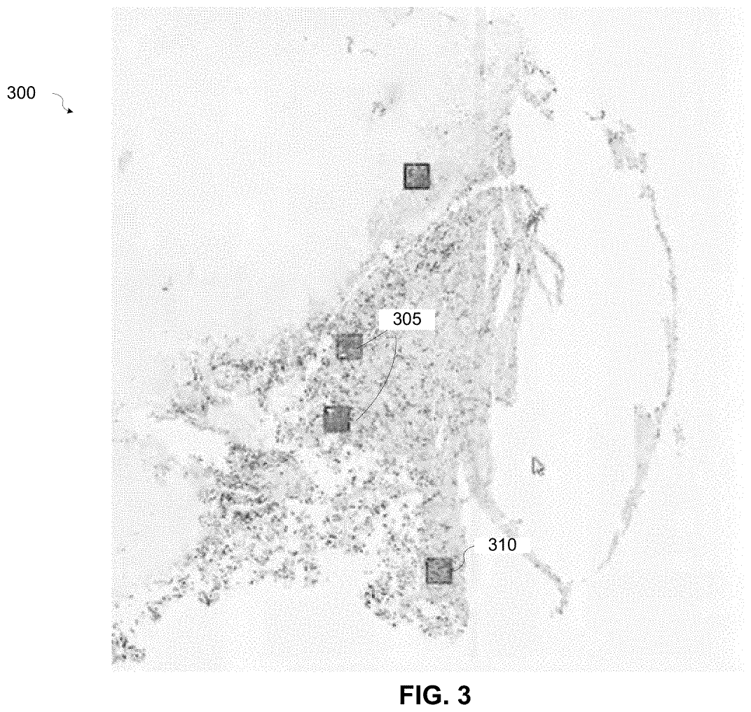

Some aspects of the present disclosure may enable identification and transfer of digital pathology annotations within selected areas of a source image to a target image. In some instances, the areas of interest may be identified on images in the image pyramid having low magnification or resolution. FIG. 3 illustrates areas of digital pathology annotations on an image of a tissue sample 300 at low magnification or resolution according to various aspects of the present disclosure. As shown in FIG. 3, the rectangles 305 may define areas of interest containing digital pathology annotations. These areas may be identified as areas requiring image analysis at higher magnification or resolution. In order to perform image analysis at higher magnification or resolution on preceding or subsequent images of the tissue sample, the identified areas, for example area 310, may be located on and transferred to the preceding or subsequent images at the low magnification or resolution. An identified area may be transferred from a source image to a target image using similar processes described herein for transferring the digital pathology annotations.

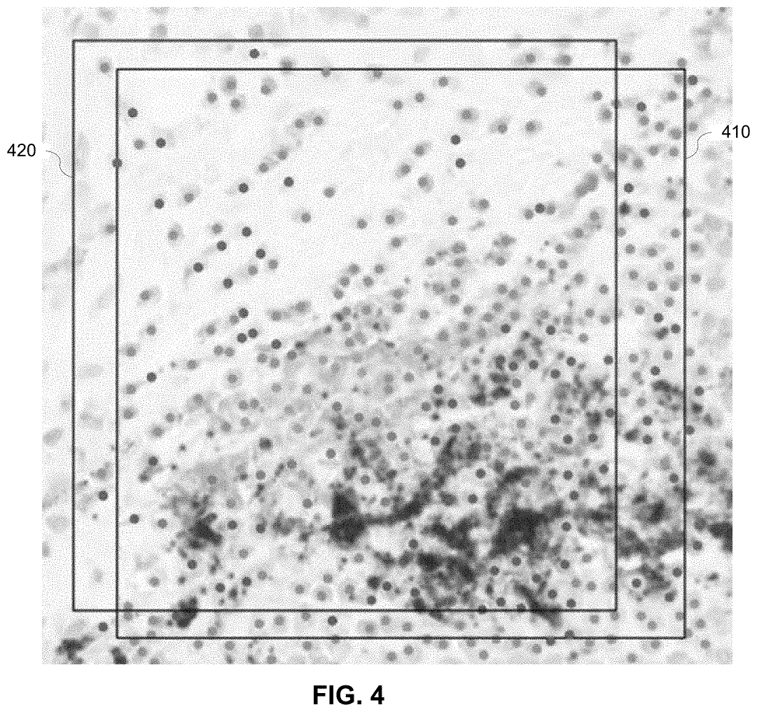

When the selected area is transferred at low magnification or resolution, misalignment between the areas of the source and target, may be negligible. However, misalignment of the digital pathology annotations within a selected area may occur that may be discernable or visible at higher magnification or resolution. FIG. 4 illustrates a misalignment of a transferred area of digital pathology annotations under high magnification or resolution according to various aspects of the present disclosure. As shown in FIG. 4, a selected area 410 containing annotations from a source image may be misaligned as shown by area 420 when transferred to the target image in low magnification or resolution. The digital pathology annotations (e.g., the specified annotations within area 410) may therefore be misaligned and visible on the target image (e.g., the area 420) when rendered at a higher magnification or resolution.

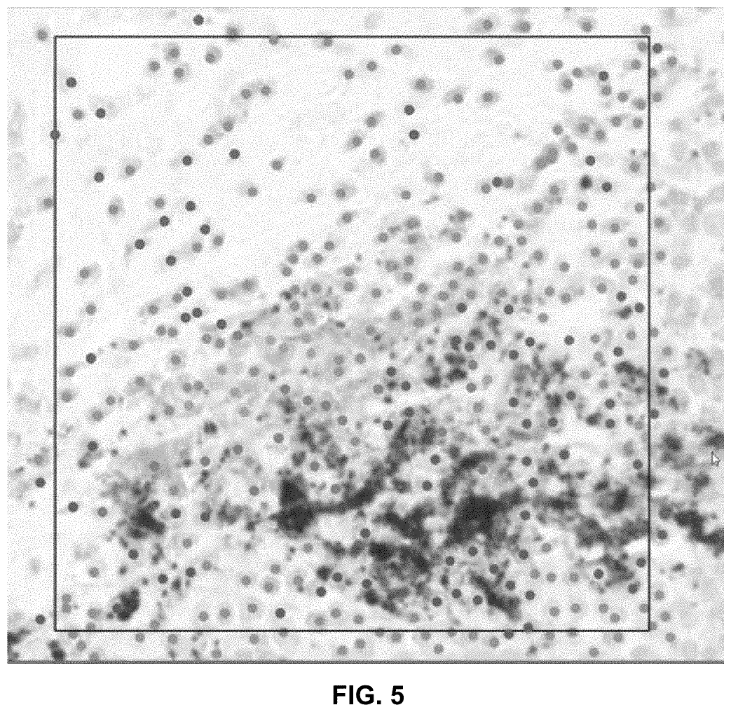

Some aspects of the present disclosure may enable alignment of the transferred areas. Referring to FIG. 4, under higher magnification or resolution, features may be identified within the selected area of the source image and the target image to establish control points within each of the rectangles. Similar to the process described herein for transferring the digital pathology annotations, pairs of matching control points between the selected areas may be identified. A transformation between the matching control points may be computed. The transformation may then be applied to the annotations within the transferred area to align the transferred area on the target image with the area of the source image. FIG. 5 illustrates the aligned transferred area of digital pathology annotations of FIG. 4 under high magnification according to various aspects of the present disclosure.

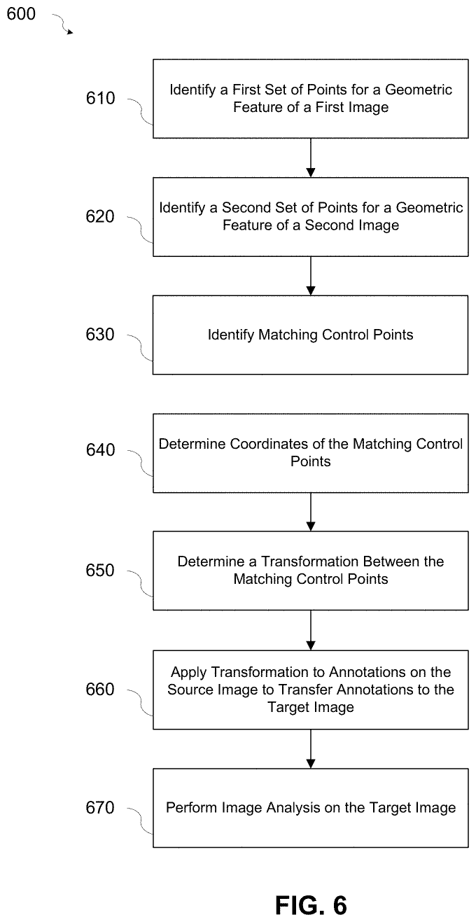

FIG. 6 is a flowchart illustrating an example of a method 600 for transferring digital pathology annotations between images according to some aspects of the present disclosure. Referring to FIG. 6, at block 610, a first set of control points are detected for a geometric feature of a first image (e.g., a source image) of a section of a tissue sample. In some instances, the first set of control points are detected within a first feature image associated with the first image.

The first feature image may be generated from an image within an image pyramid associated with the first image (e.g., a binary mask or grayscale representation of the image of the tissue section). The first image includes digital pathology annotations manually applied by a user to one or more biological structures depicted within the first image. In some instances, the image within the image pyramid is selected based on a corresponding magnification or resolution level determined between source and target tissue section images, as described in detail herein. The first feature image provides contrast between the image background and the geometric features (e.g., contour) of the tissue section. The type of feature image (e.g., greyscale or binary mask) may be selected for the first feature image by a user or selected automatically, for example, by a computer system. Control points may include distinctive aspects of the geometric features of the tissue sample, for example, corners or other pointed sections. The first set of control points detected on the feature image generated from the source image may be candidates used to locate a corresponding second set of control points on the feature image generated from the target image. A number of standard methods, for example, BRISK, SURF, FAST, etc., for detecting control points may be utilized.

At block 620, a second set of control points are detected for a geometric feature of a second image (e.g., a target image) of a preceding or subsequent section of a tissue sample. In some instances, the second set of control points is detected within a second feature image associated with the second image. The second feature image may be generated from an image within an image pyramid associated with the second image (e.g., a binary mask or grayscale representation of the image of the tissue section). The second image does not include digital pathology annotations manually applied by a user to one or more biological structures depicted within the target image. In some instances, the image within the image pyramid is selected based on the corresponding magnification or resolution level determined between source and target tissue section images, as described in detail herein. The second set of control points within the second image may be determined in the same manner as the first set of control points within the first image.

At block 630, matching control points are determined. In order to determine matching control points, features from the neighborhood of each control point within the first set of control points and the second set of control points are extracted to characterize the local appearance of each the neighborhood. The features extracted from the neighborhood of each control point may then be compared to one another to identify the closest matching (e.g., inlier) control points between the source and target images. A number of standard feature computation methods, for example, HOG, SURF, SIFT, etc., may be used for feature extraction and different matching strategies can be adopted for matching features such as threshold based matching, nearest neighbor, nearest neighbor distance ratio, and the like.

At block 640, the coordinates of the matching control points may be determined. The coordinates of the matching control points from the first set of control points with respect to the first image may be determined. The coordinates of the matching control points from the second set of control points with respect to the second image may be determined.

At block 650, a transformation matrix between the matching control points within the first image and the second image is generated using the coordinates of the matching control points. The transformation matrix provides the perspective transform of the second image with respect to the first image in terms of a fitted transformation function using translation, rotation, and scaling. Inaccuracies in matching control points can result in generation of an incorrect transformation matrix. Accordingly, in some instances, a probabilistic method or technique such as RANSAC may be utilized to filter out outlier matches and use the inlier matches to compute the transformation matrix in terms of fitting the transformation function. In some instances, image reconstruction may then be performed on the basis of the derived transformation function to align the first image with the second image. The reconstructed version of second image is then overlaid in front of the first image until all matched feature-points are overlapped. This large consolidated version of smaller images is called a mosaic or stitched image.

At block 660, the transformation matrix is applied to the digital pathology annotations within the first image to transfer the annotations to the second image on the basis of the derived transformation function. The annotations may be a set of x, y points on the first image. The transformation matrix may be a square matrix, for example, a 3×3 matrix or another size square matrix. The transformation matrix may be applied to each annotation point on the first image to obtain the transformed annotations for the second (target) image.

At block 670, image analysis may be performed on the second image. The digital pathology annotations transferred to the second image may identify the portions of the second image requiring image analysis, for example, to determine abnormal conditions such as tumor regions, necrotic regions, etc. The image analysis may be performed at the higher magnification or resolution of the second image to more accurately assess the tissue and/or cells.

It should be appreciated that the specific steps illustrated in FIG. 6 provide a particular method for transferring digital pathology annotations between images according to an embodiment of the present invention. Other sequences of steps may also be performed according to alternative embodiments. For example, alternative embodiments of the present invention may perform the steps outlined above in a different order. Moreover, the individual steps illustrated in FIG. 6 may include multiple sub-steps that may be performed in various sequences as appropriate to the individual step. Furthermore, additional steps may be added or removed depending on the particular applications. One of ordinary skill in the art would recognize many variations, modifications, and alternatives.

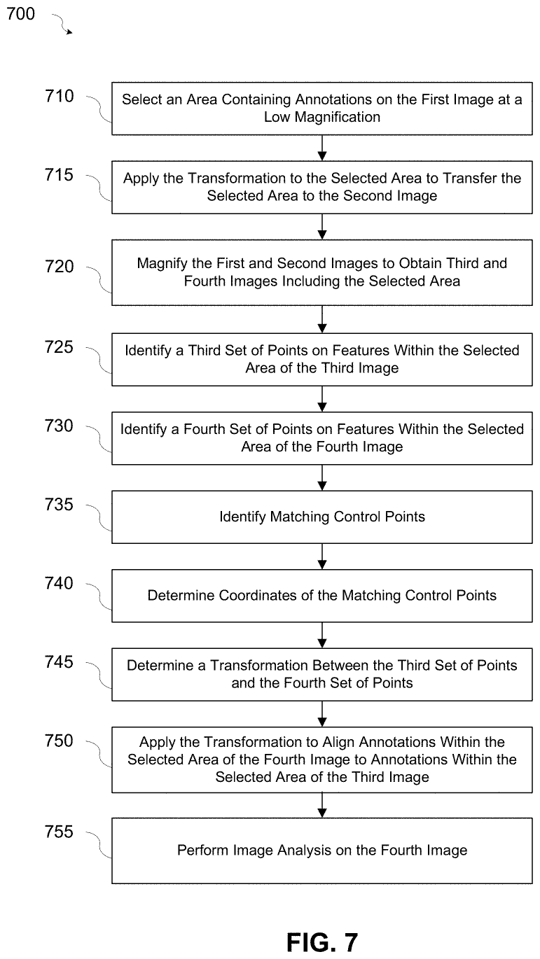

FIG. 7 is a flowchart illustrating an example of a method 700 for transferring digital pathology annotations of selected areas between images according to some aspects of the present disclosure. The method 700 of FIG. 7 may be performed after the method 600 of FIG. 6 is completed. Referring to FIG. 7, at block 710, an area containing digital pathology annotations may be selected on a first image at a low magnification. The selected area on the first (e.g., source) image may be defined by a specified shape, for example, a rectangle or other shape. The selected area may contain a large number of digital pathology annotations to be transferred to a second (e.g., target) image.

At block 715, a transformation may be applied to the selected area to transfer the selected area to a second image. The transformation for transferring the selected area from the first (e.g., source) image to the second (e.g., target) image may be computed and applied as described with respect to the method 600 of FIG. 6.

At block 720, the first (e.g., source) and second (target) images may be magnified. A higher magnification, for example, a highest available magnification, may be selected to obtain a third image of the selected area of the source image and a fourth image including the selected area of the target image. The third and fourth images may magnify the selected area to provide detail, for example, structure of the tissue sample, type and location of the annotations, etc., of the tissue sample within the selected area.

At block 725, a third set of points may be identified within the selected area of the third image. The third set of points may be control points. The control points may include distinctive aspects of the tissue sample within the selected area. For example, control points may be identified based on abnormalities in the tissue sample, specific cells, etc. In some implementations, features within the selected areas of the third image and the fourth image may be converted into a grayscale representation to provide a contrast to a background of each image. In some implementations, a binary mask may be applied to features within the selected areas of the third image and the fourth image to provide a contrast to a background of each image. The third set of points may be identify specific features based on the contrast between the background of each image and the grayscale representation or binary mask of the features A number of standard methods, for example, Block Regional Interpolation Scheme for K-Space (BRISK), Speed Up Robust Features (SURF), Features from Accelerated Segment Test (FAST), etc., for detecting control points may be utilized.

At block 730, a fourth set of points may be identified within the selected area of the fourth image. The fourth set of points may be control points. The fourth set of points on the target image may be identified in the same manner as the third set of points on the source image.

At block 735, matching control points may be identified. In order to find the corresponding control points between source and target images, features from the neighborhood of each control point may be extracted to characterize the local appearance of each the neighborhood of each control point. A number of standard feature computation methods, for example, Histogram of Oriented Gradients (HOG), SURF, Scale-Invariant Feature Transform (SIFT), etc., may be used.

At block 740, the coordinates of the matching control points may be determined. The coordinates of the matching control points from the third set of points with respect to the third image may be determined. The coordinates of the matching control points from the fourth set of points with respect to the fourth image may be determined.

At block 745, a transformation between the third set of matching control points and the fourth set of matching control points may be determined. A transformation matrix between the inlier control points on the source image and the inlier control points on the target image may be computed. Inaccuracies in matching control points can result in generation of an incorrect transformation matrix. The Random Sample Consensus (RANSAC) method may be utilized to compute the transformation matrix. The RANSAC method can filter out outlier matches and use the inlier matches to compute the transformation matrix. The transformation matrix may be a similarity transform matrix. A similarity transform matrix may provide registration (alignment) between source and target images using translation, rotation, and scaling.

At block 750, the transformation may be applied to the annotations from the selected area of the third image to transfer the annotations to the selected area of the fourth image. The transformation matrix may be applied to the annotations on the source image to transfer the annotations to the target image. Only the annotations in the selected area, rather than the entire image, may be transformed.

At block 755, image analysis may be performed on the fourth image. The digital pathology annotations transferred to the fourth image may identify the portions of the fourth image requiring image analysis, for example, to determine abnormal conditions such as tumor regions, necrotic regions, etc. The image analysis may be performed at the higher magnification or resolution of the fourth image to more accurately assess the tissue and/or cells.

It should be appreciated that the specific steps illustrated in FIG. 7 provide a particular method for transferring digital pathology annotations of selected areas between images according to an embodiment of the present invention. Other sequences of steps may also be performed according to alternative embodiments. For example, alternative embodiments of the present invention may perform the steps outlined above in a different order. Moreover, the individual steps illustrated in FIG. 7 may include multiple sub-steps that may be performed in various sequences as appropriate to the individual step. Furthermore, additional steps may be added or removed depending on the particular applications. One of ordinary skill in the art would recognize many variations, modifications, and alternatives.

The methods 600 and 700, respectively, may be embodied on a non-transitory computer readable medium, for example, but not limited to, a memory or other non-transitory computer readable medium known to those of skill in the art, having stored therein a program including computer executable instructions for making a processor, computer, or other programmable device execute the operations of the methods.

### IV. Exemplary System for Automated Image Registration

FIG. 8 is a block diagram of an example computing environment with an example computing device suitable for use in some example implementations, for example, performing the methods 600 and 700. The computing device 805 in the computing environment 800 may include one or more processing units, cores, or processors 810, memory 815 (e.g., RAM, ROM, and/or the like), internal storage 820 (e.g., magnetic, optical, solid state storage, and/or organic), and/or I/O interface 825, any of which may be coupled on a communication mechanism or a bus 830 for communicating information or embedded in the computing device 805.

The computing device 805 may be communicatively coupled to an input/user interface 835 and an output device/interface 840. Either one or both of the input/user interface 835 and the output device/interface 840 may be a wired or wireless interface and may be detachable. The input/user interface 835 may include any device, component, sensor, or interface, physical or virtual, that can be used to provide input (e.g., buttons, touch-screen interface, keyboard, a pointing/cursor control, microphone, camera, braille, motion sensor, optical reader, and/or the like). The output device/interface 840 may include a display, television, monitor, printer, speaker, braille, or the like. In some example implementations, the input/user interface 835 and the output device/interface 840 may be embedded with or physically coupled to the computing device 805. In other example implementations, other computing devices may function as or provide the functions of the input/user interface 835 and the output device/interface 840 for the computing device 805.

The computing device 805 may be communicatively coupled (e.g., via the I/O interface 825) to an external storage device 845 and a network 850 for communicating with any number of networked components, devices, and systems, including one or more computing devices of the same or different configuration. The computing device 805 or any connected computing device may be functioning as, providing services of, or referred to as a server, client, thin server, general machine, special-purpose machine, or another label.

The I/O interface 825 may include, but is not limited to, wired and/or wireless interfaces using any communication or I/O protocols or standards (e.g., Ethernet, 802.11×, Universal System Bus, WiMax, modem, a cellular network protocol, and the like) for communicating information to and/or from at least all the connected components, devices, and network in the computing environment 800. The network 850 may be any network or combination of networks (e.g., the Internet, local area network, wide area network, a telephonic network, a cellular network, satellite network, and the like).

The computing device 805 can use and/or communicate using computer-usable or computer-readable media, including transitory media and non-transitory media. Transitory media include transmission media (e.g., metal cables, fiber optics), signals, carrier waves, and the like. Non-transitory media include magnetic media (e.g., disks and tapes), optical media (e.g., CD ROM, digital video disks, Blu-ray disks), solid state media (e.g., RAM, ROM, flash memory, solid-state storage), and other non-volatile storage or memory.

The computing device 805 can be used to implement techniques, methods, applications, processes, or computer-executable instructions in some example computing environments. Computer-executable instructions can be retrieved from transitory media, and stored on and retrieved from non-transitory media. The executable instructions may originate from one or more of any programming, scripting, and machine languages (e.g., C, C++, C#, Java, Visual Basic, Python, Perl, JavaScript, and others).

The processor(s) 810 may execute under any operating system (OS) (not shown), in a native or virtual environment. One or more applications may be deployed that a include logic unit 860, an application programming interface (API) unit 865, an input unit 870, an output unit 875, a boundary mapping unit 880, a control point determination unit 885, a transformation computation and application unit 890, and an inter-unit communication mechanism 895 for the different units to communicate with each other, with the OS, and with other applications (not shown). For example, the boundary mapping unit 880, the control point determination unit 885, and the transformation computation and application unit 890 may implement one or more processes described and/or shown in FIGS. 6 and 7. The described units and elements can be varied in design, function, configuration, or implementation and are not limited to the descriptions provided.

In some example implementations, when information or an execution instruction is received by the API unit 865, it may be communicated to one or more other units (e.g., the logic unit 860, the input unit 870, the output unit 875, the boundary mapping unit 880, the control point determination unit 885, and the transformation computation and application unit 890). For example, after the input unit 870 has detected user input, may use the API unit 865 to communicate the user input to the boundary mapping unit 880 to convert a tissue section image to grayscale or apply a binary mask to the tissue section image. The boundary mapping unit 880 may, via the API unit 865, interact with the control point determination unit 885 to detect control points on the tissue section boundary. Using the API unit 865, the control point determination unit 885 may interact with the transformation computation and application unit 890 to compute and apply a transformation to digital pathology annotations of the tissue section image to transfer the digital pathology annotations to a next sequential tissue sample image.

In some instances, the logic unit 860 may be configured to control the information flow among the units and direct the services provided by the API unit 865, the input unit 870, the output unit 875, the boundary mapping unit 880, the control point determination unit 885, and the transformation computation and application unit 890 in some example implementations described above. For example, the flow of one or more processes or implementations may be controlled by the logic unit 860 alone or in conjunction with the API unit 865.

### V. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A method for transferring digital pathology annotations between images of a tissue sample, the method comprising:
identifying a first set of points for a geometric feature of a first image of a section of a tissue sample;
identifying a corresponding second set of points for a corresponding geometric feature of a second image of a same tissue sample, the second image being an image of another section of the tissue sample;
determining coordinates of the first set of points and coordinates of the second set of points;
determining a transformation between the first set of points and the second set of points; and
applying the transformation to a set of digital pathology annotations within the first image to transfer the set of digital pathology annotations from the first image to the second image.

2. The method of claim 1, further comprising:
converting an area of the section of the tissue sample for the first image and the second image into a grayscale representation to provide a contrast to a background of each image; and
identifying the geometric feature based on the contrast between the background of each image and the grayscale representation of the section of the tissue sample.

3. The method of claim 1, further comprising:
applying a binary mask to an area of the section of the tissue sample for the first image and the second image to provide a contrast to a background of each image; and
identifying the geometric feature based on the contrast between the background of each image and the binary mask of the section of the tissue sample.

4. The method of claim 1, wherein the first set of points and the second set of points contain a same number of points.

5. The method of claim 1, further comprising:
selecting an area containing a portion of the set of digital pathology annotations of the first image having a low magnification; and
applying the transformation to the selected area on the first image to transfer the selected area to a corresponding location on the second image.

6. The method of claim 5, further comprising:
magnifying the first image to a magnification higher than the low magnification to obtain a third image including the selected area;
magnifying the second image to a same higher magnification as the third image to obtain a fourth image including the selected area;
identifying a third set of points on features within the selected area of the third image;
identifying a corresponding fourth set of points on corresponding features within the selected area of the fourth image;
determining coordinates of the third set of points on the third image and coordinates of the fourth set of points on the fourth image;
determining a transformation between the third set of points and the fourth set of points; and
applying the transformation to align a set of digital pathology annotations contained in the selected area of the fourth image to the set of digital pathology annotations contained in the selected area of the third image.

7. The method of claim 6, further comprising:
extracting first features from a neighborhood of each point of the third set of points on the third image;
extracting second features from a neighborhood of each point of the fourth set of points on the fourth image; and
identifying corresponding points between the third set of points and the fourth set of points based on a comparison of the first features and the second features.

8. The method of claim 6, further comprising:
converting the features within the selected areas of the third image and the fourth image into a grayscale representation to provide a contrast to a background of each image; and
identifying specific features based on the contrast between the background of each image and the grayscale representation of the features.

9. The method of claim 6, further comprising:
applying a binary mask to the features within the selected areas of the third image and the fourth image to provide a contrast to a background of each image; and
identifying specific features based on the contrast between the background of each image and the binary mask of the section of the features.

10. The method of claim 6, wherein the third set of points and the fourth set of points contain a same number of points.

11. A system comprising:
one or more data processors; and
a non-transitory computer readable storage medium containing instructions which, when executed on the one or more data processors, cause the one or more data processors to perform actions including:
identifying a first set of points for a geometric feature of a first image of a section of a tissue sample;
identifying a corresponding second set of points for a corresponding geometric feature of a second image of a same tissue sample, the second image being an image of another section of the tissue sample;
determining coordinates of the first set of points and coordinates of the second set of points;
determining a transformation between the first set of points and the second set of points; and
applying the transformation to a set of digital pathology annotations within the first image to transfer the set of digital pathology annotations from the first image to the second image.

12. A non-transitory computer readable medium having stored therein instructions for making one or more processors execute a method for transferring digital pathology annotations between images of a tissue sample, the processor executable instructions comprising instructions for performing operations including:
identifying a first set of points for a geometric feature of a first image of a section of a tissue sample;
identifying a corresponding second set of points for a corresponding geometric feature of a second image of a same tissue sample, the second image being an image of another section of the tissue sample;
determining coordinates of the first set of points and coordinates of the second set of points;
determining a transformation between the first set of points and the second set of points; and
applying the transformation to a set of digital pathology annotations within the first image to transfer the set of digital pathology annotations from the first image to the second image.

13. The non-transitory computer readable medium as defined in claim 12, further comprising instruction for performing operations including:
converting an area of the section of the tissue sample for the first image and the second image into a grayscale representation to provide a contrast to a background of each image; and
identifying the geometric feature based on the contrast between the background of the image and the grayscale representation of the section of the tissue sample.

14. The non-transitory computer readable medium as defined in claim 12, further comprising instruction for performing operations including:
applying a binary mask to an area of the section of the tissue sample for the first image and the second image to provide a contrast to a background of each image; and
identifying the geometric features based on the contrast between the background of each image and the binary mask of the section of the tissue sample.

15. The non-transitory computer readable medium as defined in claim 12, wherein the first set of points and the second set of points contain a same number of points.

16. The non-transitory computer readable medium as defined in claim 12, further comprising instruction for performing operations including:
selecting an area containing a portion of the set of digital pathology annotations of the first image having a low magnification; and
applying the transformation to the selected area on the first image to transfer the selected area to a corresponding location on the second image.

17. The non-transitory computer readable medium as defined in claim 16, further comprising instruction for performing operations including:
magnifying the first image to a magnification higher than the low magnification to obtain a third image including the selected area;
magnifying the second image to a same higher magnification as the third image to obtain a fourth image including the selected area;
identifying a third set of points on features within the selected area of the third image;
identifying a corresponding fourth set of points on corresponding features within the selected area of the fourth image;
determining coordinates of the third set of points on the third image and coordinates of the fourth set of points on the fourth image;
determining a transformation between the third set of points and the fourth set of points; and
applying the transformation to align a set of digital pathology annotations contained in the selected area of the fourth image to the set of digital pathology annotations contained in the selected area of the third image.

18. The non-transitory computer readable medium as defined in claim 17, further comprising instruction for performing operations including:
extracting first features from a neighborhood of each point of the third set of points on the third image;
extracting second features from a neighborhood of each point of the fourth set of points on the fourth image; and
identifying corresponding points between the third set of points and the fourth set of points based on a comparison of the first features and the second features.

19. The non-transitory computer readable medium as defined in claim 17, further comprising instruction for performing operations including:
converting the features within the selected areas of the third image and the fourth image into a grayscale representation to provide a contrast to a background of each image; and
identifying specific features based on the contrast between the background of each image and the grayscale representation of the features.

20. The non-transitory computer readable medium as defined in claim 17, further comprising instruction for performing operations including:
applying a binary mask to the features within the selected areas of the third image and the fourth image to provide a contrast to a background of each image; and
identifying specific features based on the contrast between the background of each image and the binary mask of the section of the features.
